(19) United States
(12) Patent Application Publication
(10) Pub. No.: US 2011/0039293 A1
(43) Pub. Date: Feb. 17, 2011

(54) Effervescent Solid Pharmaceutical Composition Comprising Dextrose and Process for its Preparation

(76) Inventor: Juan J. Hernandez Sierra, Bogota (CO)

Correspondence Address: HOLLAND & HART, LLP, P.O BOX 8749, DENVER, CO, 80201, US

(21) Appl. No.: 12/600,225

(22) PCT Filed: May 19, 2008
(86) PCT No.: PCT/IB2008/001346

§ 371 Date: Sep. 29, 2010

(30) Foreign Application Priority Data
May 15, 2007 (CO) 07-048,367

(51) Int. Cl. C12Q 1/02

(52) U.S. Cl. 435/29

(57) ABSTRACT

The present invention is related to the medicine field. Specifically, the present Invention is related with an effervescent solid pharmaceutical composition of dextrose having as purpose determining the metabolized glucose levels in a patient with diabetes or glucose metabolism related conditions, in a simple and effective manner with respect to the conventional products of this kind. Accordingly, the present invention provides a novel product allowing the medical professional or physician of a clinic laboratory to determine in a quantitative, economic, efficient, fast and in a single manner, the clinical conditions of a patient regarding the glucose level and the pancreas ability to eliminate the unnecessary sugars in terms of time. Furthermore, the present invention provides a novel process for preparing the novel effervescent solid pharmaceutical formulation of dextrose.

The present invention is related to the medicine field. Particularly, the present invention is related with a novel product allowing the medical professional to determine the glucose levels and the pancreas ability to eliminate the unnecessary sugars in terms of time. Furthermore, the present invention provides a novel process for preparing said pharmaceutical composition.

### BACKGROUNDS OF THE INVENTION

In the state of the art is well known that blood glucose levels (also named blood sugar levels) are indications about how the diabetes is well controlled and how effective the care schedule is working (diet, exercise and medicament). If the blood sugar levels are consistently under control (with levels almost normal), the complications of diabetes can be prevented or its progress may be made lowered. Those people with diabetes have check its blood sugar levels up four times at day. Sugar levels may be affected vary several factors, including the following:
Diet.
Diabetes Drugs.
Exercise.
Stress.
Diseases.

Generally, the blood sugar levels checking and the pancreas ability to eliminate the unnecessary sugars are very important in the appropriate handling of diabetes. However, the actual methods for measuring blood sugar require a blood sample; and this blood sugar may be measured in home using many invader instruments in order to obtain a blood sample (invader means penetrating the body tissue with a medical instrument).

Usually, a drop of blood obtained by means of a pinprick in the finger is sufficient for using a test strip which is subsequently measured in a monitor. This pinprick in the finger may be made with a little lancet (a special needle) or with an instrument having its lancet over a spring, which quickly prick finger tip. The blood drop is located on the test strip. The test strip is then located on special monitor for blood glucose (also called glucose measurer) which read the blood glucose levels. However, this type of techniques do not make possible curves for quantitatively determining the efficiency of pancreas and insulin versus time, since these techniques only offer one detailed measurement of glucose. Furthermore, this practice is uncomfortable to the patient; even more if several measurements are needed by day.

Now well, up today are commercially available many types of monitors, which vary in price, easily of use, size, portability and test durability. Each of this monitor requires its own test strips. In addition, it is well known that the blood glucose monitors are precise and reliable if are correctly used, and the majority provides results in a few minutes. Some of these glucose monitor may provide instructions and results verbally for those persons having visual or physical disabilities. There are also glucose monitors having verbal instructions in Spanish or other languages.

Certain monitors for blood glucose determination are equipped with information handling systems, which means that the blood sugar measurement is automatically registered every time in the memory. Some medical offices have computerized systems compatible with this information handling systems, which allow the transfer of the blood sugar levels records and other information electronically. One advantage of the information handling system is the capability to make a curve by representing the standard blood sugars levels. However, these systems may result very expensive and unavailable for certain users or for certain communities.

Accordingly, it s clear that exists an urgent need of a novel alternative that help medical professionals to determine in a quantitative, economic, efficient, rapid and a single manner, the blood glucose levels in the patient or the pancreas ability to eliminate the unnecessary sugars in terms of time, and overcome thus the disadvantages existing with the conventional systems, previously discussed. In this sense, none document of the state of the art had been suggested a practical and feasible solution allowing the medical professional the detection of blood glucose levels or the pancreas capability to eliminate the unnecessary sugars in terms of the time. In this manner, in the searching of an urgent solution to this existing problem, and after extended investigative process, the applicant made possible a novel product that, in addition to the overcoming of the industry disadvantages, is highly desirable to the patient and the medical professional due its simplicity, economy, fast and efficiency for quantitatively determining the blood sugar levels and the pancreas capability to eliminate the unnecessary sugars.

### DETAILED DESCRIPTION OF THE INVENTION

In order to overcome the above exposed disadvantages, the present invention provides a novel product which, due to its specific components and proportions, IS an effervescent product of easy dilution in cold water, ready for use, remarkably ease the medical or physician of a clinical laboratory the quantitative determination of glucose levels and the pancreas ability to eliminate unnecessary sugars in a patient affected by said conditions. Therefore, the target patient avoids undesirable pinpricks; the ingestion of undesirable solutions due the low solubility of the active ingredient; or the undesirable waiting in the determination of curve due to the time expensive by extended dilution solutions.

Particularly, the present invention provides an effervescent solid pharmaceutical composition, characterized by comprising as essential components:
(a) An hydrating agent,
(b) An acidifier agent,
(c) An alkalinizing agent,
(d) A flavoring agent,
(e) A coloring agent.

Regarding the hydrating agent, it may be selected, for example, from the group consisting of dextrose, sucrose, fructose, lactose, maltose, mannitol, xilitol and mixtures thereof.

Preferably, in one preferred embodiment of the present invention, the effervescent solid pharmaceutical composition is characterized by the hydrating agent is dextrose.

In one preferred embodiment, the effervescent solid pharmaceutical composition is characterized by said hydrating agent is present in the pharmaceutical composition in an amount varying from 25 to 99% by the total weight of the composition.

More preferably, the hydrating agent is present in the pharmaceutical composition of the present invention in an amount varying from 50 to 99% by the total weight of the composition.

Regarding the acidifier agent present in the pharmaceutical composition of the present invention, it may be selected, for example, from the group consisting of citric acid, tartaric acid, malic acid, fumaric acid and mixtures thereof.

In one preferred embodiment of the present invention, the effervescent solid pharmaceutical composition comprises citric acid as the acidifier.

In order to reach an appropriate pH, said acidifier agent may be present in the effervescent solid pharmaceutical composition of the present invention in an amount varying from 0.1 to 40% by the total weight of the composition.

Even more preferred is that the acidifier agent is present in an amount which may vary from 2 to 10% by the total weight of the pharmaceutical composition of the present invention.

Regarding the alkalinizing agent present in the pharmaceutical composition of the present invention, it may be selected, for example, from the group consisting of sodium bicarbonate, potassium bicarbonate, sodium citrate, potassium citrate, calcium carbonate, sodium phosphate and mixtures thereof.

In one preferred embodiment of the present invention, the pharmaceutical composition of the present invention is characterized by the alkalinizing agent is sodium bicarbonate.

This alkalinizing agent is present in an amount varying from 1 to 70% by the total weight of the pharmaceutical composition of the present invention.

Even more preferably, the alkalinizing agent is present in an amount that may vary from 2 to 30% by the total weight of the composition of the present invention.

On the other hand, the pharmaceutical composition of the present invention is characterized by the flavoring agent may be selected, for example, from the group consisting of Orange, Mandarin, Lemmon, Cherry, Strawberry, Tuttifruti, Grape, Peach, raspberry and mixtures thereof.

In a preferred embodiment, the effervescent solid pharmaceutical composition of the present invention is characterized by the flavoring agent is Orange flavor.

On the other hand, the effervescent solid pharmaceutical composition of the present invention is characterized by said flavoring agent is present in the pharmaceutical composition in an amount varying from 0.2 to 30% by the total weight of the composition. Preferably, the flavoring agent is present in the pharmaceutical composition in an amount that may vary from 1 to 10% by the total weight of the composition of the present invention.

Regarding the coloring agent that may be present in the effervescent solid pharmaceutical composition of the present invention, it may be selected from the group consisting of Blue, Yellow, Red, Orange, Green and mixtures thereof.

According to other embodiment of the effervescent solid pharmaceutical composition of the present invention is characterized by the coloring agent is Yellow.

Thus, in order to provide an appropriate color, said coloring agent is present in an amount that may vary from 0.001 to 1% by the total weight of the pharmaceutical composition of the present invention. More preferably, the coloring agent is present in an amount that may vary from 0.05 to 0.5% by the total weight of the composition.

The effervescent solid pharmaceutical composition according to the present invention may be presented in diverse dose forms with the purposes. For example, the composition of the present invention may be presented in the form of an effervescent powder, a granulate powder, a suspension powder, a syrup, micro granules, tablets, coated tablets, chewable tablets or gelatin capsules.

In one preferred embodiment, the effervescent solid pharmaceutical composition of the present invention is presented as an effervescent powder.

Effervescent powders are formulations in which composition generally takes part acid substances and carbonates or bicarbonates able to quickly react in presence of water evolving carbon dioxide. They are addressed to be dissolved or be dispersed in water before its administration. In this manner, the effervescent powders are dissolved in water at time of its administration in order to occur the acid-base reaction (citric acid-sodium bicarbonate), and thus to form carbonic gas which help to hide or enhance product flavors having undesirable flavor of some pharmaceutical products. These products should be packaged into entirely hermetically recipients in order to prevent that an acid-base reaction be produced by the environmental humidity. Sodium bicarbonate and citric acid form anti acids due its effervescent reaction with water. They reduce the acidity of stomach fluids, since that there is an excess that neutralizes. This help to reveal the heat stomach symptoms and indigestion.

In this manner, the effervescent pharmaceutical forms as those of the present invention having in its composition an acid substance and a basic substance, such as carbonate or bicarbonate, have the property to evolve carbonic gas, which favors the pharmaceutical form disintegrating (e.g., tablet, powder) and the immediate releasing of the active substance in order to perform its pharmacologic action.

In one preferred embodiment, the present invention provides an effervescent solid pharmaceutical composition, characterized by it specifically comprises the dextrose as the hydrating agent, citric acid as the acidifier agent, sodium bicarbonate as the alkalinizing agent, Orange flavor as the flavoring agent and Yellow as the coloring agent.

In one preferred embodiment, the effervescent solid pharmaceutical composition of the present invention is characterized by the hydrating agent is dextrose, the acidifier agent is citric acid, the alkalinizing agent is sodium bicarbonate, the flavoring agent is Orange flavor and the coloring agent is Yellow, and it is in the form of granules.

In this manner, the inventive aspect of the product of the present invention relies on the fact that its specific components and proportions make it an effervescent product of easy dilution in cold water, ready for use, that significantly ease to the medical personnel the quantitative determination of glucose levels and the pancreas capability for eliminating unnecessary sugars in a patient affected by such conditions. Accordingly, the novel product of the present invention allows the making of quantitative curves of glucose levels versus time in fast, simple and economic manner. This had not been possible up today with the current techniques, since they only allow the detection of sugar levels in certain point; and they also required administering to the patient products of lower solubility as to start said evaluation, which is absolutely undesirable both patient as the medical personnel. In the market there is not available an effervescent product of dextrose allowing to ease the evaluation process and detection of glucose levels in a patient and the pancreas capability for eliminating the unnecessary sugars.

In other object, the present invention provides a process for preparing the effervescent solid pharmaceutical composition of the present invention, characterized by comprising the following steps:
(i) Powdering the acidifier agent,
(ii) Mixing the hydrating agent, the acidifier agent, the alkalinizing agent and the flavoring agent.
(iii) Drying of mixture of above step (ii)
(iv) Controlling the relative humidity content (% RH), bottling and packaging.

In one preferred embodiment of the process of the present invention the mixing step of the hydrating agent, the acidifier agent, the alkalinizing agent, the flavoring agent and the coloring agent allows that said mixture being conformed as granules having an average size varying from 250 to 6000 um.

And more preferably, the granules conformed in the mixing step have a particle size comprised varying from 400 to 4000 um.

Consequently, the process involve as initial step the weighting of raw materials including the hydrating agent, the acidifier agent, the alkalinizing agent, the flavoring agent, and the coloring agent. A verification of said weighting is made and said weighted materials are to the mixing step. This mixing may be carried out, for example, in a V-type mixer, but the person skilled in the art would understand that it is possible the use of other type of mixers allowing the suitable mixing of the raw materials for the purposes of the present invention. Subsequently, it shall proceed to the powdering of the acidifier agent, which may be citric acid for the instant illustrative example of preparation. This powdering may be carried out, for instance, in a Fitzmil Miller, but other milling equipment may be used with similar characteristics. Next, the dry mixing of all of the raw material, which includes dextrose, sodium bicarbonate, citric acid, orange flavor, and yellow coloring in the present case. This mixing is carried out in the V-type mixer and over 30 minutes. After this time, the mixture is placed into dryness oven for drying the mixed product. This drying step may be carried out at a temperature of about 40° C. during about 8 hours. Once the drying step had concluded, the quality control tests are performed over the product, which include determination of relative humidity (% RH); and the corresponding physicochemical tests. Once satisfactorily concluded these test, it shall proceed to the bottling and packaging of product for its distribution.

Thus, the product of the present invention may be administered to the patient in the form of a unique oral dose of glucose as solution form for determining in a quantitative, fast, economic and simple manner the capability to metabolize glucose in a patient with sugar levels related conditions. Therefore, the test allows to the medical professional determine diabetes mellitus, gestational diabetes, hyperinsulin and any other investigative study involving diabetic patients, once the patient had ingested the effervescent product of dextrose of the present invention.

Accordingly, once administered the novel product of the present invention to the patient, it should proceed with the assays measuring the capability to metabolize the glucose. Persons affected by diabetes mellitus have high glucose levels in blood and the tolerance assays to the glucose are one of tools for its diagnosis. This assay is also performed in order to diagnose diabetes mellitus in researching studies involving diabetic and in the cases, where is suspected the presence of this disease, although a fast blood glucose test had been made, with normal results; as well as for the hyperinsulin diagnostic (increasing of insulin levels).

Methods more currently used in order to evaluate the tolerance may be:
(i) Tolerance tests by using an oral single dose of glucose.
(ii) Tolerance tests with an intravenous dose of glucose.

More commonly glucose tolerance test is oral. After a fasting night, the patient ingests a solution containing a known amount of glucose. Basal blood sample is taken, before the patient ingest the glucose solution, and again each 30 minutes latter up to 2 or 3 hours according to the medical prescription, for determining glycemia. In addition, the patient cannot eat during the exam and it is recommended to inform the medical professional about the use of drugs that may affect the results.

Frequently is applied for measuring the insulin levels (hormone produced by the pancreas which allows introducing glucose from the blood to each one of the body cells).

When glucose is orally administered, the absorption from gastrointestinal tract toward blood continues during a variable lapse, which depends upon the amount of the administered glucose. Maximal glucose absorption is estimated in 0.8 g/kg weight by hour.

Tolerance to the glucose orally administered, measure the balance between the passage of glucose through extra cell fluid and its separation by cell assimilation and the urinary excretion, if exists.

Therefore, test may be affected, not only by those factors involved with the use of glucose, but also by those affecting its absorption.

Intravenous glucose tolerance tests are not common. In order to perform this assay, the patient is intra venous injected with a known amount of glucose during three minutes, previous to the measure of blood insulin levels at minute one and minute three.

### Tolerance Test By Using An Oral Dose:

Theoretically, during 3 days previous to the performance of the test, the patient is administered with a diet containing about 300 g of carbohydrates and about 3000 calories. Previous fasting should be from 8 to 9 hours. Glucose doses used are 75 g. In general, standard prepared and flavored solutions are used. The solution should be cool. Venous blood is collected previous the glucose ingestion and every half hour, or each hour, during 3 hours after of glucose ingestion, according the medical prescription for determining glycemia, and simultaneously for glucose levels.

### Interpretation

At normal conditions, the blood should have a glucose level lower tan 100 mgl/dl. The normal blood values are:
Ayunas: 60 a 100 mg/dl.
1 hour: less than 200 mgldl.
2 hours: less tan 140 mg/dl.
Between 140 and 199 is considered that glucose intolerance exists and is a group having higher risk to develop diabetes
Levels above 200 mgl/dl, indicates diabetes diagnostic.

Criteria used for defining the abnormality condition of a tolerance curve are based on the peak level reached by the blood concentration and the absence of return to the normal level, 2 hours after glucose intake, where the last is the more important.

A hypoglucemic value (i.e., lowered glycemia) of 3 to 5 hours after the glucose ingestion was observed in certain patients, whose the tolerance curve was diabetic, understood as hyperinsulin, which is typical of a diabetic condition.

### EXAMPLE

The following is an illustrative example for only one specific formulation of the present invention:
Product: Effervescent Dextrose
Pharmaceutical Form: Non esterile powder
Doisification weight: Sachet×25 grams
Measurement Unit of Product: kilogram

| Material Description | Amount per Unit | Measurment Unit |
|---|---|---|
| MONOHYDRATED | 900.2500000 | G |
| DEXTROSE | | |
| POWDER | | |
| YELLOW No. 6 FDC | 0.1000000 | G |
| ORANGE FLAVOR | 9.6500000 | |
| POWDER | | |
| SODIUM BICARBONATE | 40.0000000 | G |
| USP | | |
| ANHYDRIC CITRIC ACID | 50.0000000 | G |
| USP | | |

The following corresponds to the physicochemical results obtained from a sample representative of the active principle contained in a pharmaceutical formulation of the present invention:

### Monohydrated Dextrose Rosferose

| DESCRIPTION | WHITE POWDER, | |
|---|---|---|
| | ODORLESS, SWEET FLAVOR, | |
| | WATER SOLUBLE | |
| PRODUCT CLEANING ASPECT | | ACCORD |
| IDENTIFICATION TEST | | ACCORD |
| IN SOLUTION ASPECT | | 2 |
| ROTATORY POWER | DEGREES | +52.9 |
| ACIDITY ML NAOH 0.020N/5 G | ML | <0.300 |
| DRYNESS LOSS | % | 8.9 |
| SULFATED ASHES | % | <0.10 |
| CHLORIDES | PPM | <5.0 |
| SULFATES | PPM | <10.0 |
| ARSENIC | PPM | <1.00 |
| HEAVY METALS | PPM | <5.0 |
| OTHER SUGARS, DEXTRINES | | 2 |

### Advantages of the Invention

Particularly, the advantages of the claimed product and process, and the scope representing such advantages, may be based on of the following considerations:
It is a low cost product.
Allow the fast and effective detection of the glucose levels.
It is a product having excellent tolerance for the patient, since the products as effervescent pharmaceutical form had demonstrated higher bioavailability and are less aggressive at gastric and stomach level.
It is easy and comfortable for the patient due its application simplicity (unique oral intake).
The product provides time saving for the medical professional or practicing of a clinical laboratory due the easy dissolution of dextrose at the moment for preparing the patient dose. Some products commercially available should dissolve in warm water for its subsequent cooling, while the product of the present invention is effervescent and is dissolved in instantaneous form.
In order to preserve the content and the potentially effervescent effect of the product of the present invention in each sachet, it is necessary to use high impermeability barrier materials, which increases the product protection against possible contamination and the durability of the product is increased in comparison with the commercial products.
The target patient that intakes the product of the present invention avoid undesirable pinpricks; the ingestion of undesirable solutions due the low solubility of the active component; or the undesirable waiting in the determination of the curve due time expensive with extended dilution solutions.

Now well, any person skilled in the art, particularly any person skilled who had access to the teachings of the present invention shall recognize without major difficulty that it is possible any modification over the product or process disclosed therein, without the same departure from the scope and spirit of the invention. For instance, it would be recognized that any variety of ingredients that comply with the purposes of the invention or any proportions may be used. Consequently, all of the embodiments and modifications exposed in the present invention should not be understood as limitations of the scope of the invention, which is defined by the contents of the following claims.

1. Effervescent Solid pharmaceutical composition for quantitatively determining the glucose levels and the pancreas ability to eliminate unnecessary sugars, CHARACTERIZED BY comprising:
(a) An hydrating agent,
(b) An acidifier agent,
(c) An alkalinizing agent,
(d) A flavoring agent,
(e) A coloring agent.

2. Effervescent Solid pharmaceutical composition according to claim 1, characterized by the hydrating agent may be selected from the group consisting of dextrose, sucrose, fructose, lactose, maltose, mannitol, xilitol and mixtures thereof.

3. Effervescent Solid pharmaceutical composition according to claim 2, characterized by the hydrating agent is dextrose.

4. Effervescent Solid pharmaceutical composition according to claim 1, characterized by said hydrating agent is present in the pharmaceutical composition in an amount varying from 25 to 99% by the total weight of the composition.

5. Effervescent Solid pharmaceutical composition according to claim 1, characterized by said hydrating agent is present in the pharmaceutical composition in an amount varying from 50 to 99% by the total weight of the composition.

6. Effervescent Solid pharmaceutical composition according to claim 1, characterized by the acidifier agent may be selected from the group consisting of citric acid, tartaric acid, malic acid, fumaric acid and mixtures thereof.

7. Effervescent Solid pharmaceutical composition according to claim 6, characterized by the acidifier is citric acid

8. Effervescent Solid pharmaceutical composition according to claim 1, characterized by said acidifier agent is present in the pharmaceutical composition in an amount varying from 0.1 to 30% by the total weight of the composition.

9. Effervescent Solid pharmaceutical composition according to claim 8, characterized by said acidifier agent is present in the pharmaceutical composition in an amount varying from 2 to 10% by the total weight of the composition.

10. Effervescent Solid pharmaceutical composition according to claim 1, characterized by the alkalinizing agent may be selected from the group consisting of sodium bicarbonate, potassium bicarbonate, sodium citrate, potassium citrate, calcium carbonate, sodium phosphate and mixtures thereof.

11. Effervescent Solid pharmaceutical composition according to claim 10, characterized by the alkalinizing agent is sodium bicarbonate.

12. Effervescent Solid pharmaceutical composition according to claim 1, characterized by said alkalinizing agent is present in the pharmaceutical composition in an amount varying from 2 to 70% by the total weight of the composition.

13. Effervescent Solid pharmaceutical composition according to claim 12, characterized by said alkalinizing agent is present in the pharmaceutical composition in an amount varying from 2 to 30% by the total weight of the composition.

14. Effervescent Solid pharmaceutical composition according to claim 1, characterized by the flavoring agent may be selected from the group consisting of Orange, Mandarin, Lemmon, Cherry, Strawberry, Tuttifruti, Grape, Peach, raspberry and mixtures thereof.

15. Effervescent Solid pharmaceutical composition according to claim 14, characterized by the flavoring agent is Orange flavor.

16. Effervescent Solid pharmaceutical composition according to claim 1, characterized by said flavoring agent is present in the pharmaceutical composition in an amount varying from 0.5 to 30% by the total weight of the composition.

17. Effervescent Solid pharmaceutical composition according to claim 16, characterized by said flavoring agent is present in the pharmaceutical composition in an amount varying from 1 to 10% by the total weight of the composition.

18. Effervescent Solid pharmaceutical composition according to claim 1, characterized by the coloring agent may be selected from the group consisting of Blue, Yellow, Red, Orange, Green and mixtures thereof.

19. Effervescent Solid pharmaceutical composition according to claim 18, characterized by the coloring agent is Yellow.

20. Effervescent Solid pharmaceutical composition according to claim 1, characterized by said coloring agent is present in the pharmaceutical composition in an amount varying from 0.001 to 1% by the total weight of the composition.

21. Effervescent Solid pharmaceutical composition according to claim 20, characterized by said coloring agent is present in the pharmaceutical composition in an amount varying from 0.05 to 0.5% by the total weight of the composition.

22. Effervescent Solid pharmaceutical composition according to claim 1, characterized by the hydrating agent is dextrose, the acidifier agent is citric acid, the alkalinizing agent is sodium bicarbonate, the flavoring agent is Orange flavor and the coloring agent is Yellow.

23. Effervescent Solid pharmaceutical composition according to claim 1, characterized by said pharmaceutical composition is an effervescent powder, a granulate powder, suspension powder, syrup, micro granules, tablets, coated tablets, chewable tablets or gelatin capsules.

24. Effervescent Solid pharmaceutical composition according to claim 23, characterized by said pharmaceutical composition is an effervescent powder.

25. Effervescent Solid pharmaceutical composition according to claim 1, characterized by the hydrating agent is dextrose, the acidifier agent is citric acid, the alkalinizing agent is sodium bicarbonate, the flavoring agent is Orange flavor and the coloring agent is Yellow, and it is as granules.

26. Process for preparing an effervescent solid pharmaceutical composition according to any of preceding claims, characterized by comprising:
(i) Powdering the acidifier agent,
(ii) Mixing the hydrating agent, the acidifier agent, the alkalinizing agent and the flavoring agent.
(iii) Drying of mixture of above step (ii)
(iv) Controlling the relative humidity content (% RH), bottling and packaging.

27. Process according to claim 26, characterized by the mixing step of the hydrating agent, the acidifier agent, the alkalinizing agent, the flavoring agent and the coloring agent conform granules having an average size varying from 250 to 6000 um.

28. Process according to claim 27, characterized by the granules having a particle size comprised varying from 400 to 4000 um.

29. Process according to claim 26, characterized by the step of mixing the ingredients is carried out in a V-type mixer during 30 minutes.

30. Process according to claim 26, characterized by the step of drying the mixed product is carried out during 8 hours at a temperature of 40° C.
